(19) United States
(12) Patent Application Publication
(10) Pub. No.: US 2019/0167917 A1
(43) Pub. Date: Jun. 6, 2019

(54) SYSTEMS, METHODS AND APPARATUSES FOR USE WITH SUBCUTANEOUS IMPLANTS

(71) Applicant: Advent Access Pte. Ltd., Singapore (SG)

(72) Inventors: Ruey Feng PEH, Singapore (SG); Jo Ern YEOH, Singapore (SG); Henry SUSAINATHAN, Singapore (SG); Hongye YE, Singapore (SG); Iris Siu Kwan TAN, Singapore (SG)

(73) Assignee: Advent Access Pte. Ltd., Singapore (SG)

(21) Appl. No.: 16/203,468

(22) Filed: Nov. 28, 2018

Related U.S. Application Data
(60) Provisional application No. 62/591,702, filed on Nov. 28, 2017.

(51) Int. Cl. A61M 5/42; A61M 39/02

(52) U.S. Cl. CPC A61M 5/425; A61M 2209/088; A61M 39/0208

(57) ABSTRACT

Systems, apparatuses and methods for stabilizing an implant used in a medical procedure, for example, an implant used to facilitate repeated needling in kidney dialysis. The apparatus may include an implant holder including at least one implant-engaging surface configured to cause compression of skin adjacent the implant. The apparatus may also include a holding mechanism coupled to the implant holder and configured to engage patient anatomy in order to secure the implant holder in place above the implant. In certain implementations, the implant holder may be coupled to an adhesive patch or may be flexible and configured to at least partially conform to the patient anatomy when a strap assembly is tightened around patient anatomy.

### RELATED APPLICATION(S)

This application claims priority to and the benefit of U.S. Provisional Application No. 62/591,702, filed Nov. 28, 2017, which is hereby incorporated by reference.

### BACKGROUND

Numerous medical conditions require repeated treatments, for example, repeated needling or cannulation for blood draws, dialysis, administration of drugs, etc. Medical implants may be used in conjunction with such treatments, for example, to guide a needle to a particular location. These medical implants may be placed just under the skin so that they can be easily accessed. In some cases, treatment can be delivered by medical practitioners that are trained to locate and utilize the implant; in other cases, treatments may be performed wholly or in part by the patient.

### SUMMARY

Systems, methods and apparatuses are disclosed that may be utilized for stabilizing an implant (e.g., a medical implant) as may be used, for example, during a kidney dialysis procedure. In certain embodiments, the apparatus can include an implant holder including at least one implant-engaging surface configured to cause compression of skin adjacent an implant. The apparatus may also include a holding mechanism, such as a strap assembly, coupled to the implant holder and configured to tighten around patient anatomy in order to secure the implant holder in place above the implant.

The implant holder can further include side walls where the implant-engaging surfaces are on the side walls. Some variations of the implant holder can include a tightening mechanism configured to cause movement of the side walls, thereby adjusting a compressive force around the implant. In other variations, the side walls can be fixed.

In one implementation, a slot can be formed in the implant holder such that the implant holder is substantially open above the implant. An implant holder may also include an aperture having an inner surface with a shape complementary to the shape of the implant.

In other implementations, the implant holder may be flexible and configured to at least partially conform to the patient anatomy when a strap assembly is tightened around the patient anatomy. Such flexing of the implant holder can cause movement of its side walls to increase a compressive force around the implant.

An implant holder can also include legs extending from it having feet at the ends of the legs, where the feet are configured to frictionally engage with the skin of the patient and pull the skin taut when the implant holder is flattened against the patient anatomy.

In other implementations, an implant holder may have an aperture that includes an implant-engaging surface and the implant holder may be flexible and shaped to form a space between the implant holder and the patient such that when the implant holder is at least partially flattened against a patient anatomy, a vacuum forms in the space to hold the implant holder in place.

Further implementations disclosure herein may have an open configuration where implant-engaging elements are disposed around opposing sides of the implant and a closed configuration where the implant-engaging elements cause a compression of skin adjacent the implant that stabilizes the implant. The implant holder can also include a retaining mechanism that maintains the compression. Such apparatuses can also include an adhesive patch for adhering the implant holder to the skin of the patient, where the implant holder is coupled to the adhesive patch.

In other embodiments, the implant holder can be configured to change from an open configuration to a closed configuration by application of force and plastic deformation of the implant holder.

Also disclosed is an exemplary method that can include placing of an implant holder coupled with an adhesive patch, in its open configuration, at a location adjacent the implant by adhering the adhesive patch to the skin of the patient. A force can be applied to the implant holder to cause the implant holder to go into the closed configuration and stabilize the implant. A medical procedure can be performed using the stabilized implant. The implant holder and the adhesive patch can then be removed from the skin of the patient. In some cases, the patient can perform one or more of these method steps.

In still other embodiments, an apparatus can include a holding mechanism that includes one or more of: a strap assembly, an elastic band, an adhesive patch, a suction cup, an articulated arm, a vise-like mechanism, or a press mechanism.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to particular implementations, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A is a diagram illustrating a simplified side-sectional view of a portion of a patient about to undergo a needling procedure in conjunction with an implant, as may be performed in accordance with certain aspects of the present disclosure,

FIG. 1B is a diagram illustrating a simplified front-sectional view of the portion of the patient including the implant,

FIG. 2A is a diagram illustrating a simplified side-sectional view of FIG. 1A with an implant holder compressed onto the patient's anatomy in accordance with certain aspects of the present disclosure,

FIG. 2B is a diagram illustrating a simplified front-sectional view of FIG. 1B with an implant holder compressed onto the patient's anatomy in accordance with certain aspects of the present disclosure,

FIG. 3A is a diagram illustrating a simplified top view of an implant holder located above an implant and held in place by a strap assembly in accordance with certain aspects of the present disclosure,

FIG. 3B is a diagram illustrating a simplified side view of an implant holder located above an implant and held in place by an articulated arm in accordance with certain aspects of the present disclosure,

FIG. 3C is a diagram illustrating a simplified side view of an implant holder located above an implant and held in place by an open vise mechanism in accordance with certain aspects of the present disclosure,

FIG. 3D is a diagram illustrating a simplified side view of an implant holder located above an implant and held in place by a closed vise mechanism in accordance with certain aspects of the present disclosure,

FIG. 3E is a diagram illustrating a simplified side view of an implant holder located above an implant and held in place by a press mechanism in accordance with certain aspects of the present disclosure,

FIG. 4 is a diagram illustrating a simplified perspective view of an implant holder including an end wall in accordance with certain aspects of the present disclosure,

FIG. 5 is a diagram illustrating a simplified perspective view of an implant holder including a slot in accordance with certain aspects of the present disclosure,

FIG. 6 is a diagram illustrating a simplified perspective view of an implant holder having an aperture and skin-stretching legs in accordance with certain aspects of the present disclosure,

FIG. 7A is a diagram illustrating a simplified perspective view of an implant holder having a flexible construction in accordance with certain aspects of the present disclosure,

FIG. 7B is a diagram illustrating a simplified perspective view of an additional implant holder having a flexible construction in accordance with certain aspects of the present disclosure,

FIG. 8 is a diagram illustrating a simplified perspective view of an implant holder having movable side walls and a tightening knob in accordance with certain aspects of the present disclosure,

FIG. 9 is a diagram illustrating a simplified bottom view of the implant holder of FIG. 8 in accordance with certain aspects of the present disclosure,

FIG. 10 is a diagram illustrating a simplified exploded view of an implant holder with movable side walls and a locking knob in accordance with certain aspects of the present disclosure,

FIG. 11A is a diagram illustrating a simplified perspective view and front elevational view of an implant holder having movable side walls and a locking screw in accordance with certain aspects of the present disclosure,

FIG. 11B is a diagram illustrating a simplified side-sectional view of the implant holder of FIG. 11A in accordance with certain aspects of the present disclosure,

FIG. 12 is a diagram illustrating a simplified perspective view of an exemplary implant holder configured to be held in place by suction between the implant holder and the patient in accordance with certain aspects of the present disclosure,

FIG. 13 is a diagram illustrating a simplified side-sectional view of the exemplary implant holder of FIG. 12,

FIG. 14 is a diagram illustrating a simplified bottom-perspective view of a portion of the exemplary implant holder of FIG. 12,

FIG. 15 is a diagram illustrating a simplified view of an adhesive patch used in conjunction with an implant holder in an open configuration in accordance with certain aspects of the present disclosure,

FIG. 16 is a diagram illustrating a simplified view of the implant holder of FIG. 15 in a closed configuration,

FIG. 17 is a diagram illustrating a simplified view of an exemplary wire implant holder in an open configuration in accordance with certain aspects of the present disclosure,

FIG. 18 is a diagram illustrating a simplified view of the exemplary wire implant holder of FIG. 17 in a closed configuration, and

FIG. 19 is a diagram illustrating a method in accordance with certain aspects of the present disclosure.

### DETAILED DESCRIPTION

Systems, methods and apparatuses are described herein for use in conjunction with medical procedures, for example, medical procedures that make use of an implant within a patient. The technologies described herein may be used with any number of medical procedures. In one particular example, kidney dialysis is performed utilizing subcutaneous implants that help guide needles toward particular locations in a patient's blood vessels.

Because medical implants are often located within soft tissues of a patient (e.g., implanted subcutaneously), they are capable of inadvertent movement during medical procedures. Thus, it can be beneficial for an implant to be stabilized and/or properly positioned before use. For example, an implant that guides a needle to a blood vessel can be stabilized such that the guide is properly located and oriented in order to allow the needle to reliably reach the blood vessel. As such, the present disclosure provides systems, methods and apparatuses that can aid in stabilizing implants that may be used by medical practitioners or by individuals performing medical treatments on themselves.

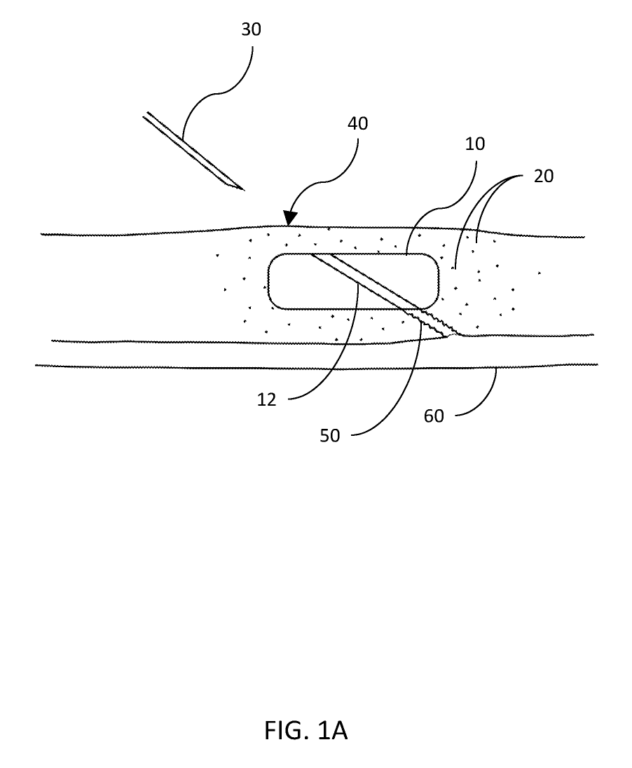

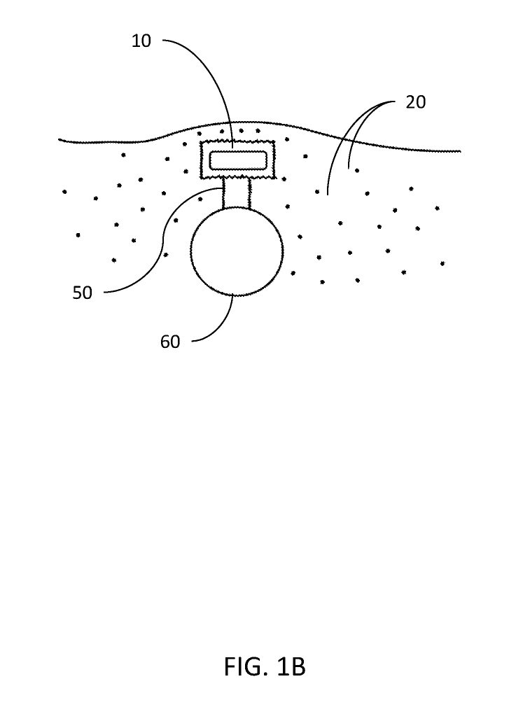

To provide an exemplary illustration of a procedure utilizing an implant, reference is made to FIGS. 1A (side view) and 1B (end view) showing an implant 10 located in skin 20 that can assist with guiding a needle 30 during a medical procedure. This exemplary procedure may be used for a patient undergoing kidney dialysis and can allow needling to occur at repeatable location, which can prolong the life of the vessel being repeatedly accessed.

In the example of FIG. 1A, implant 10 is shown having guide passageway 12 that is shaped to accept needle 30. In use, the needle punctures the patient's outer skin surface 40 and goes into the upper portion of guide passageway 12. The needle then continues through the implant and into tract 50 (e.g., a tract in the patient's skin that may be formed and/or surrounded by scar tissue due to fibrosis). Due to the guiding of the implant, the needle can enter vessel 60 at the proper location and the proper angle.

While the example of needling for a dialysis procedure is used, the technologies disclosed herein are contemplated to be useful in other medical procedures, with other implant designs, and in conjunction with other parts of a patient's anatomy.

In some cases, an implant may be somewhat unstable or mobile, for example, because it is located in a patient's soft tissue that can be stretched, moved or compressed. In addition, certain implants can be small or difficult to locate or access. The technologies of the present disclosure can, among other things, help to stabilize implants so that medical procedures using the implants can be properly performed. Stabilization may be achieved, for example, by using an implant holder to compress skin around an implant. As used herein, the term “stabilize” means to reduce, substantially reduce, or completely eliminate at least one of a translation or a rotation of an implant.

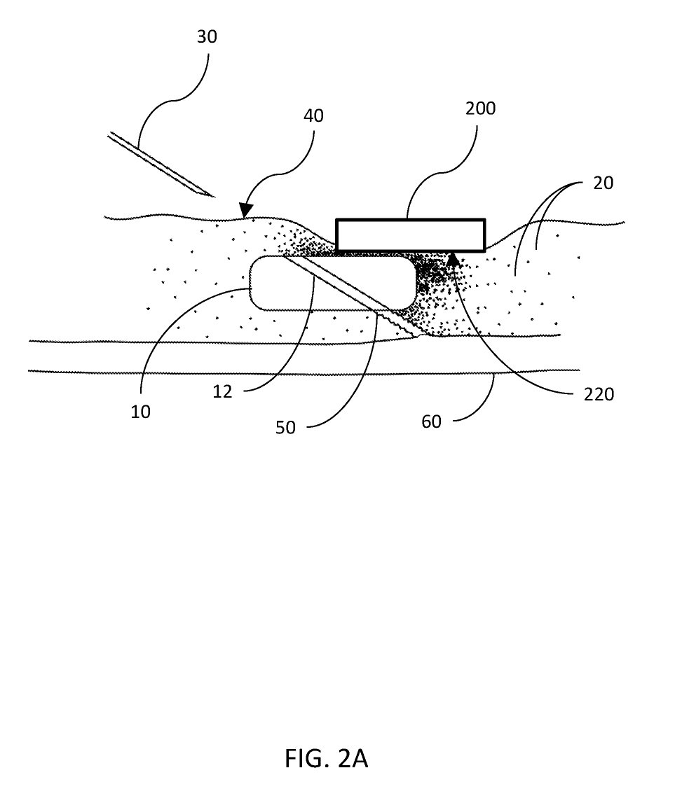

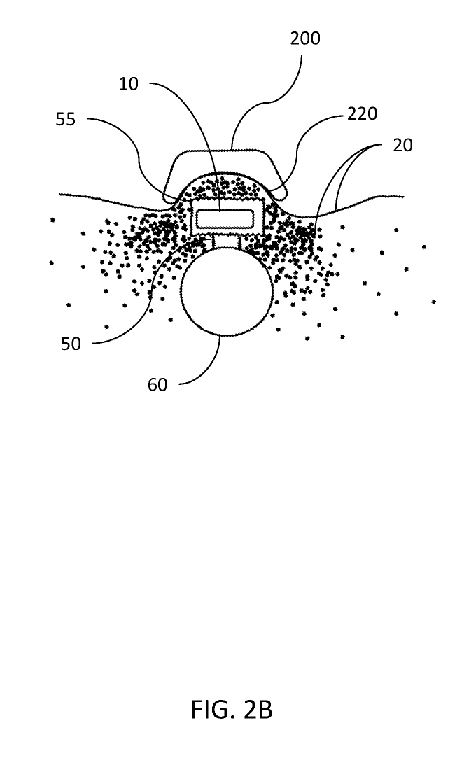

FIGS. 2A (side view) and 2B (end view) depict an exemplary apparatus for stabilizing an implant of a patient using tissue compression effected through the implant holder. Implant holder 200 can include at least one implant-engaging surface 220 configured to cause compression of skin 20 adjacent the implant. Implant holder 200 can be positioned above implant 10 and then pressed onto the patient's skin causing a compression of the skin adjacent the implant. The compression of the skin adjacent the implant is illustrated in an exemplary fashion in FIGS. 2A and 2B by the densification of the dot pattern in the skin adjacent the implant (as compared to the less dense pattern shown in FIGS. 1A and 1B).

As used herein, the term “implant-engaging surface” refers to any surface or surfaces configured to cause compression of skin adjacent an implant when an implant holder is being used. An example of an implant-engaging surface is shown in the simplified example of FIG. 2B by implant-engaging surface 220.

As used herein, “skin adjacent an implant” means a tissue volume at least partially surrounding or near the implant. Skin adjacent the implant can include, for example, a tissue volume on one or more sides of an implant, on the top or bottom of the implant, at an end of the implant, etc. Also, as used herein, “on top” or “above” an implant means generally located in the direction of the skin surface relative to the implant.

Finally, as used herein, “skin” is used in a broad manner to refer to any tissue that an implant can be disposed in including muscle, etc. (although when reference is made to the “skin surface” it is understood that this refers to the outer surface of the patient's skin).

The implant holders discussed herein can be secured in place above an implant, for example, by being held by a patient, by being held by a person assisting a patient, or by a particular holding mechanism. When held in place by a person, the implant holder can be held, for example, by a hand, or one or more fingers, that may also apply a force to the implant holder to cause compression of skin adjacent the implant.

When an implant holder is held in place by a holding mechanism, the holding mechanism can be used to locate the implant holder above the implant and it may also be configured to apply particular forces to the implant holder and the implant. Furthermore, when a holding mechanism is used, such may free up a patient's hand to facilitate self-treatment utilizing the implant.

Exemplary holding mechanisms include straps (or strap assemblies, as described further below), clamps, elastic bands, adhesive patches, etc. While the present disclosure describes a number of different holding mechanisms in conjunction with particular implementations, it is contemplated that these holding mechanisms may be utilized with other implant holder designs and that other holding mechanisms not described may also be used to secure an implant holder in place.

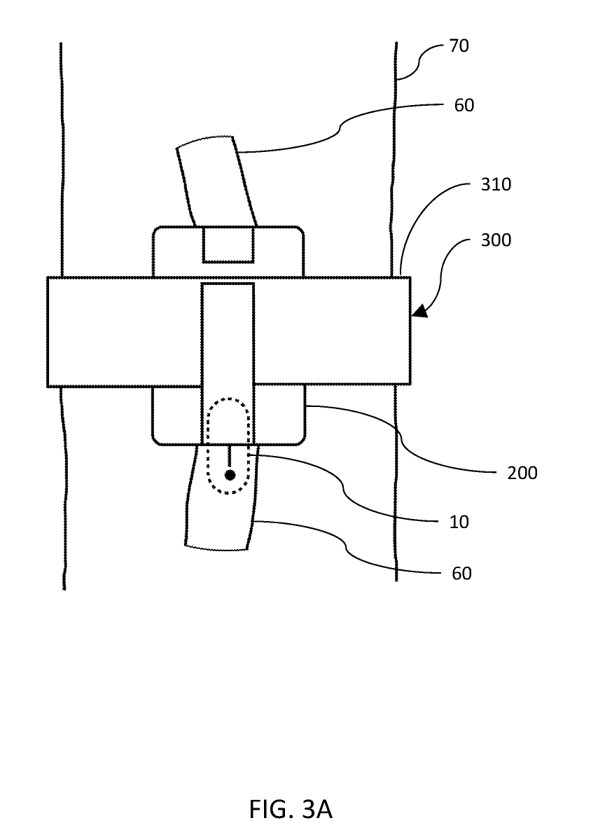

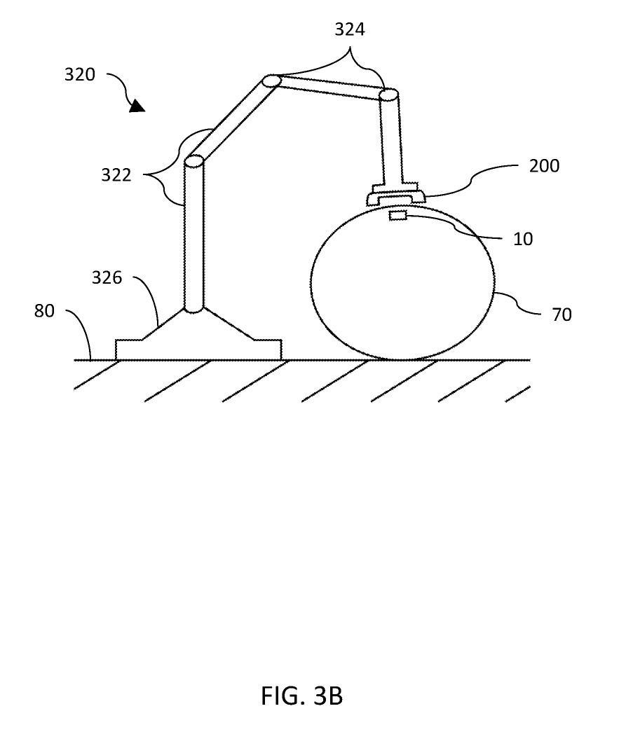

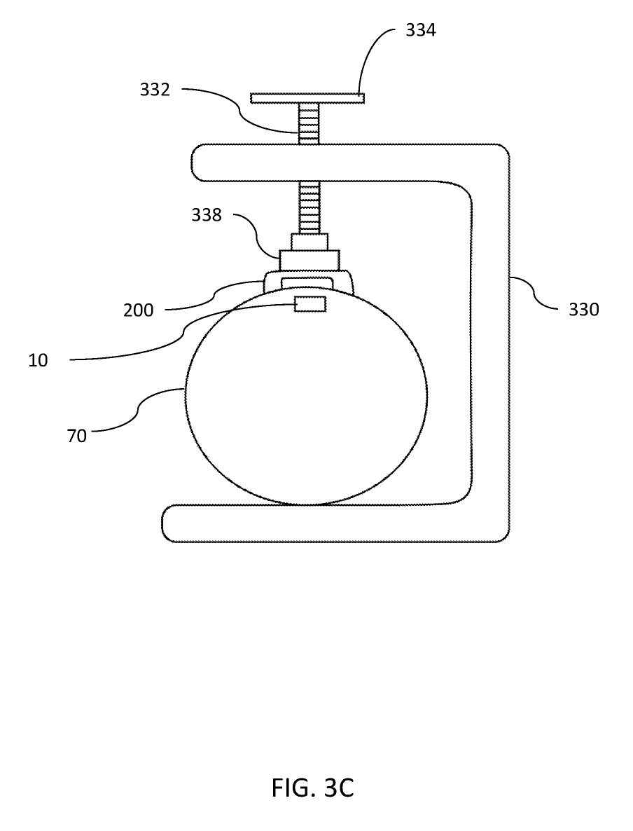

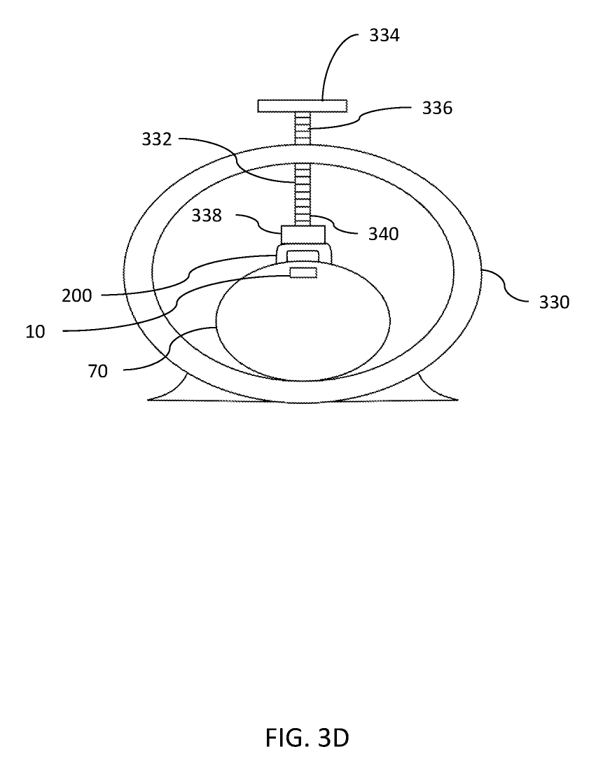

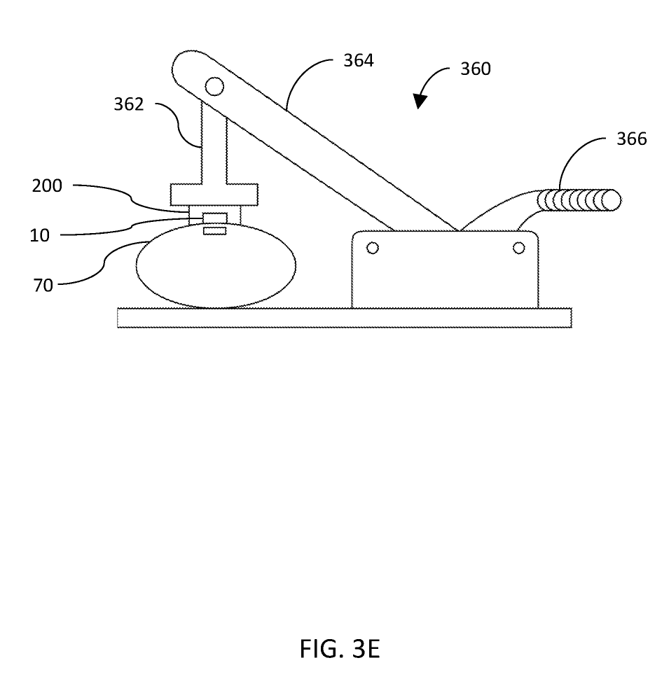

As shown in FIG. 3A, one example of a holding mechanism can be strap assembly 300. A strap assembly can be coupled to the implant holder and configured to tighten around patient anatomy 70 (e.g., an arm, a leg, etc.) in order to secure the implant holder in place above the implant. Strap assembly 300 can include not only the strap shown, but also securing, tightening, releasing components, etc. For example, a strap assembly can include material with hooks and loops (like Velcro), holes and buckles (similar to a belt/watch), clasps with teeth to hold the strap when tightened, etc. The strap itself can be made of flexible fibrous material, an elastic band, leather, cloth, etc. The strap can be generally flat in cross-section or can be thinner and more rope-like.

Alternative holding mechanisms are shown in FIGS. 3B-E. In the implementation of FIG. 3B, an articulated arm can be used to position an implant holder relative to the patient's anatomy 70 and implant 10. The patient's arm 70 is shown resting on solid surface 80. Next to the patient's arm is an articulating arm 320 that includes a number of sections 322 connected by joints or hinges 324. The articulating arm can be adjusted at the hinges to put the implant holder at the proper location and angle so that a desired compression may be achieved. To maintain compression, the hinges can be tightened or can have an integrated retaining mechanism (e.g., an inherent tightness in the hinge) to prevent the hinges from moving unless a sufficient external force is applied. The articulated arm can be attached to the surface with a base 326 that is connected to one of the sections. The base can be a solid base (e.g., such that it could be bolted or screwed to the solid surface). The base could also be, for example, a suction cup that holds the articulated arm to the solid surface.

In the alternative implementations of FIGS. 3C and 3D, the holding mechanism can be a vise-like structure that is used to secure the implant holder in place. In these examples, the patient's anatomy can be placed inside a rigid frame 330 supporting implant holder 200. The rigid frame 330 can include a threaded rod 332 with a handle 334 and a grip 338. The implant holder 200 can be placed next to or affixed to the grip 338. The threaded rod can then be turned by the handle such that the threaded rod advances through the rigid frame and pushes the implant holder toward the patient's anatomy compressing the implant holder onto the patient and around the implant. In the example of FIG. 3C, the rigid frame can be open on one side to allow lateral access by the patient anatomy. In the example of FIG. 3D, the rigid frame can be closed and the patient's anatomy can then enter the rigid frame in a generally axial direction.

In yet another implementation, shown in FIG. 3E, a press mechanism 360 can be used to provide the location and compression of the implant holder. Similar to the vise-like implementations shown in FIGS. 3C and 3D, the patient's anatomy can be placed at the proper location underneath a grip or pad supporting implant holder 200. The grip or pad can be connected to a vertical section 362 that is itself connected to a lever section 364. The lever section can then in turn be connected at a fulcrum to a handle section 366 such that when the handle section is actuated, the lever section advances downward applying a compressive force to implant holder 200 via vertical section 362.

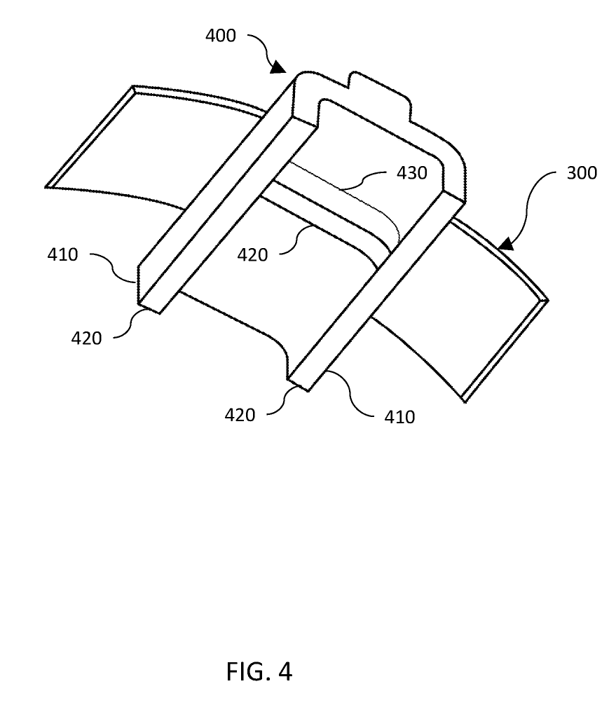

The present disclosure provides many exemplary implant holder designs that can assist with implant stabilization. One implementation is shown in FIG. 4 where implant holder 400 includes side walls 410 that have implant-engaging surfaces 420. In some implementations, these side walls can be fixed (i.e., not able to move). Also, the implant holder may also include an end wall 430 disposed between the side walls for resisting an axial motion of the implant (e.g., by compressing the skin at an axial location from the implant holder). With the examples used herein, and with additional reference to FIG. 3A, axial motion (or axial direction) is understood to mean the motion or direction generally following the direction of the blood vessel (which, in FIG. 3A, is aligned with the long axis of the implant). This is distinct from “lateral” or “side-to-side” motions or directions which are considered to be generally (but not necessarily exactly) perpendicular to the axial direction.

In use, the exemplary implant holder of FIG. 4 can be positioned above an implant and a strap assembly can be used to tighten implant holder onto the patient anatomy. Side walls 410 and end wall 430 can then compress the patient tissue adjacent the implant, thereby reducing the possible axial and/or lateral movement of the implant during the medical procedure.

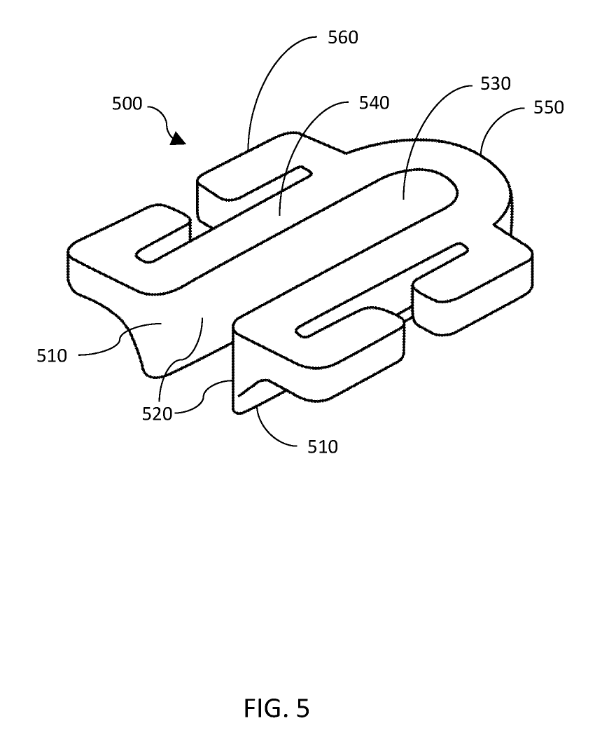

Another implementation, shown in FIG. 5, can include an implant holder 500 with side walls 510 forming slot 530 such that the implant holder is substantially open above the implant. In this exemplary implementation, the implant holder 500 can have a body 540 with side walls 510 extending downward (toward the patient anatomy when deployed). Also, the body and side walls may be shaped into a “U-shape” by including a connecting section 550. The connecting section can also fix the distance between the two side walls (thereby fixing the width of slot 530). Also shown are buckles 560 that can be connected to a strap so that when the strap is tightened, the implant holder pushes down onto the patient anatomy causing compression of the skin adjacent the implant by implant-engaging surfaces 520.

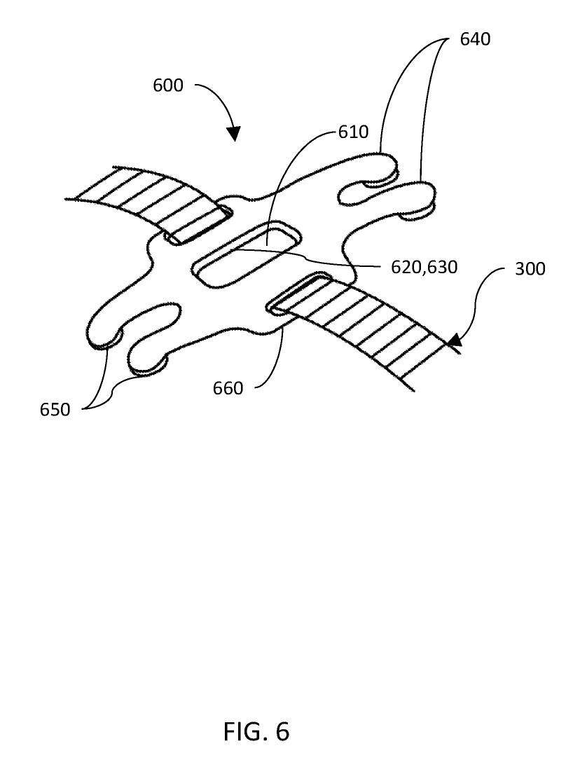

In another implementation, illustrated in FIG. 6, implant holder 600 can include an aperture 610 having an inner surface 620 that has a complementary shape to the implant. In this example, inner surface 620 comprises the implant-engaging surface 630. This implementation is conceptually similar to that described in FIG. 5 in that when the implant holder is tightened onto the patient's anatomy, the implant (or the skin adjacent the implant) can at least partially come up into the aperture and engage the inner surface. As used herein, the term “complementary shape” means a shape that is similar to that of an implant. The complementary shape can be larger or smaller than the implant shape and may have some design differences (e.g., rounded corners vs sharp corners, etc.).

Implementations of implant holders described herein may include additional design features that result in a pulling of the skin surface near the implant to provide additional stabilization. For example, with reference to the exemplary implementation shown in FIG. 6, at least a portion of implant holder 600 can be flexible and configured to at least partially flatten against the patient anatomy. Flattening may be performed, for example, by tightening a strap assembly coupled to implant holder 600 at buckle 660. Such an implant holder may include legs 640 extending from the implant holder, the legs having feet 650 at the ends of the legs and the feet being configured to frictionally engage with the skin of a patient and to pull the skin taut when the implant holder is flattened. This tightening of the skin can have the effect of additionally compressing the skin around the implant, limiting movement, and improving stabilization of the implant.

Embodiments of the present disclosure are contemplated to include any number of legs and/or feet (e.g., 0, 1, 2, 3, 4, 6, 8, etc.), and the legs and/or feet can have any shape. The legs can be elongate, can be straight or curved, and can be configured so that when the implant holder is pulled down by the strap assembly, the legs bend to more closely conform to the shape of the patient anatomy. In certain embodiments, the legs need not be flexible, but can merely be designed to press on the skin and stretch it when the implant holder is secured in place against the patient anatomy. When the present disclosure refers to “legs” in the plural, such also contemplates designs that include only a single structure, rather than a dual/bifurcated structure as shown in FIG. 6.

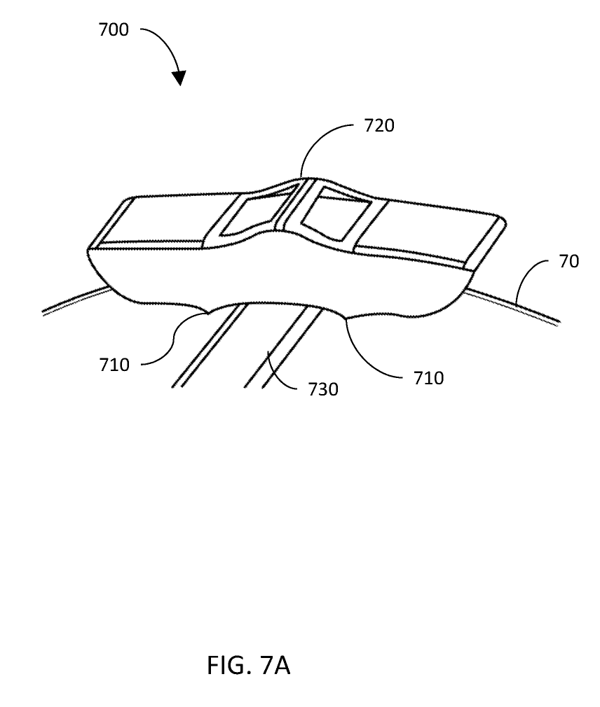

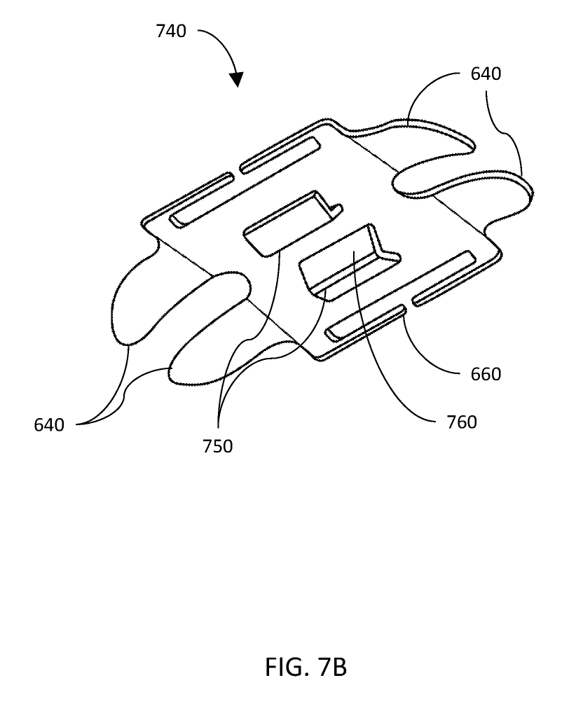

The feet can be of any shape, (e.g., circular, oval, square, etc.) and can be constructed of a separate material suitable for frictional engagement with skin (e.g., rubber, felt, plastic, etc.). It is also contemplated that the feet are not a separate material but simply the ends of the legs 640 (as shown in the example of FIG. 7B).

In the particular implementation shown in FIG. 6, there are two pairs of two legs, each of the legs having a separate material foot for frictional engagement with the patient's skin. While the legs may be oriented in the axial direction as shown, they can also be oriented in other directions such as laterally, or in directions between axially or laterally.

In the implant holder implementation depicted in FIG. 7A, the implant holder 700 can be flexible and configured to at least partially conform to patient anatomy 70 when a strap assembly is tightened around the patient anatomy 70. In the example of FIG. 7A, a strap assembly can be coupled with implant holder 700 through a buckle 720. Of course, other methods for securing the implant holder are contemplated, as discussed herein.

Such a flexible implant holder design can be configured so that the flexing of the implant holder causes movement of side walls 710 in order to increase a compressive force around the implant. It can be seen from FIG. 7A that flexing of the implant holder to more closely conform to patient anatomy will cause side walls 710 to move closer to the location of the implant 730.

FIG. 7B illustrates another implementation of a flexible implant holder 740. Similar to the implant holder 700 in FIG. 7A, when the implant holder 740 is flattened against a patient's anatomy, side walls 750 can engage the implant. In this particular implementation, the sidewalls 750 are reoriented so that the implant engaging surfaces 760 form a shape more complementary to the implant and cause compression of the skin adjacent the implant. In one implementation, the implant holder of FIG. 7B may be flattened by the tightening of a strap that couples with buckles 660.

Similar to the embodiment of FIG. 6, the particular implementation of FIG. 7B also includes legs 640 that may be configured to stretch the skin around the implant for further stabilization.

In certain embodiments of the present disclosure, implant holders can include mechanisms to allow for the movement and fixation of side walls to provide compression of skin adjacent the implant.

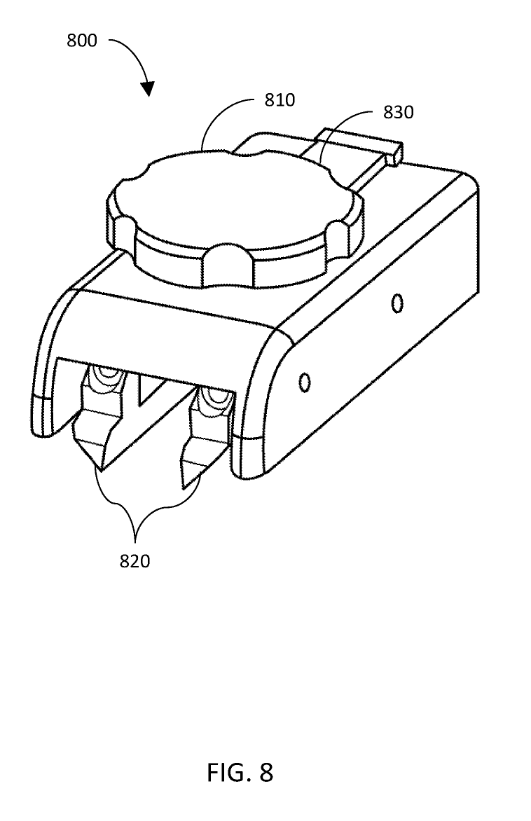

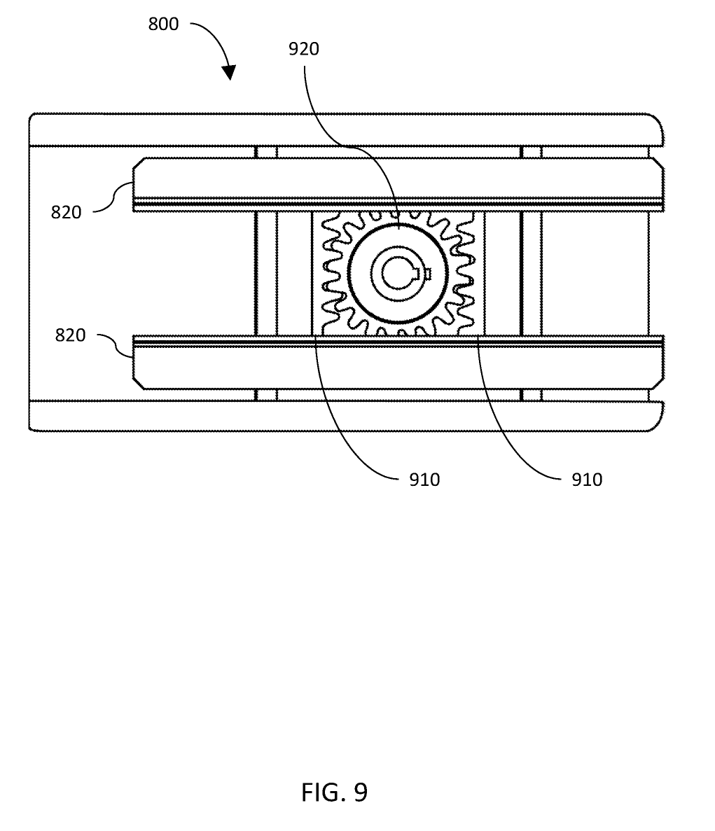

In one exemplary implementation illustrated in FIG. 8 (perspective view) and FIG. 9 (bottom view), an implant holder 800 can include a tightening mechanism configured to cause movement of side walls 820 thereby adjusting a compressive force around the implant. As used herein, the term “tightening mechanism” is understood to mean a mechanism that can cause a tightening (or loosening) of some components of the implant holder (e.g., movement of side walls inward/outward) to adjust the compression of skin adjacent an implant.

As shown in FIG. 9, an exemplary tightening mechanism can include gear tracks 910 coupled to side walls 820 and a gear 920 configured to engage the gear tracks and cause the movement of the side walls. The tightening mechanism can also have a knob 830 (shown in FIG. 8) coupled to gear 920 and configured such that actuation of the knob causes movement of the side walls.

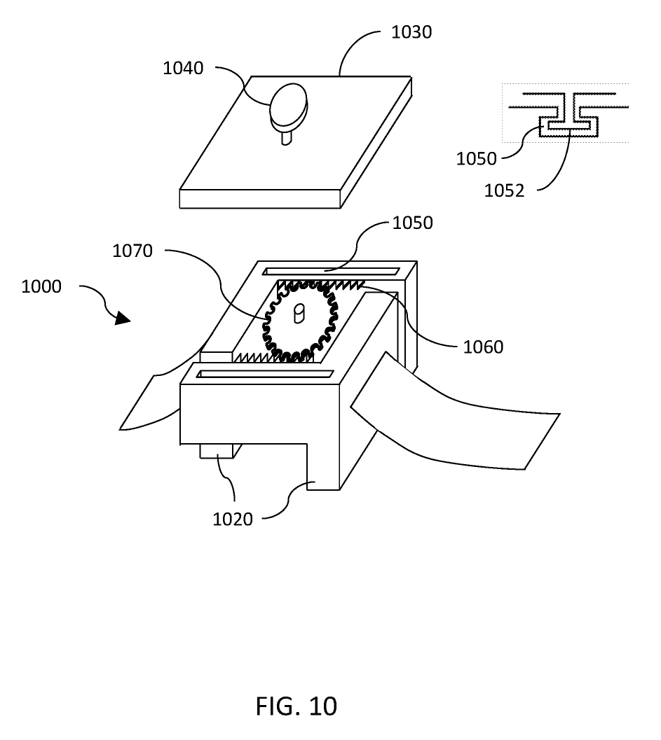

In an additional implementation, illustrated in FIG. 10, two side walls 1020 are configured to move relative to each other such that they can be tightened about the implant. Controlled movement can be facilitated through the use of, for example, rod tracks 1050 and rods 1052 that can be included in lid 1030. Similar to the embodiment of FIG. 9, there can also be a gear 1070 and gear tracks 1060 formed in the side wall structures to cause movement of the walls. The tightening mechanism can also include a lock knob 1040 in communication with the gear and configured such that, when in a locked position, the lock knob locks the gear in place and prevents the movement of the side walls. In some implementations, such locking can be performed by including in the lid a gear-shaped recess that can accept the gear when lock knob 1040 is pulled in a vertical direction. When the gear engages the gear-shaped recess, it can no longer be turned, thereby locking side walls in place.

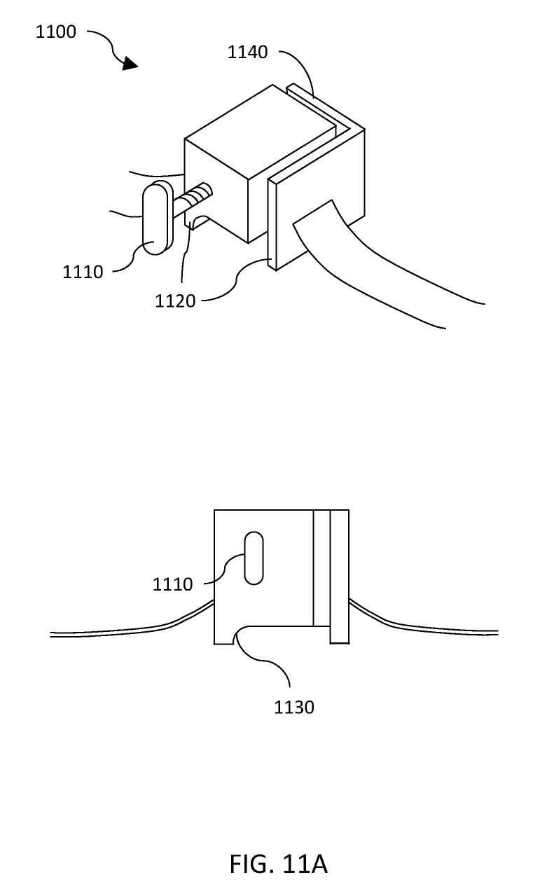

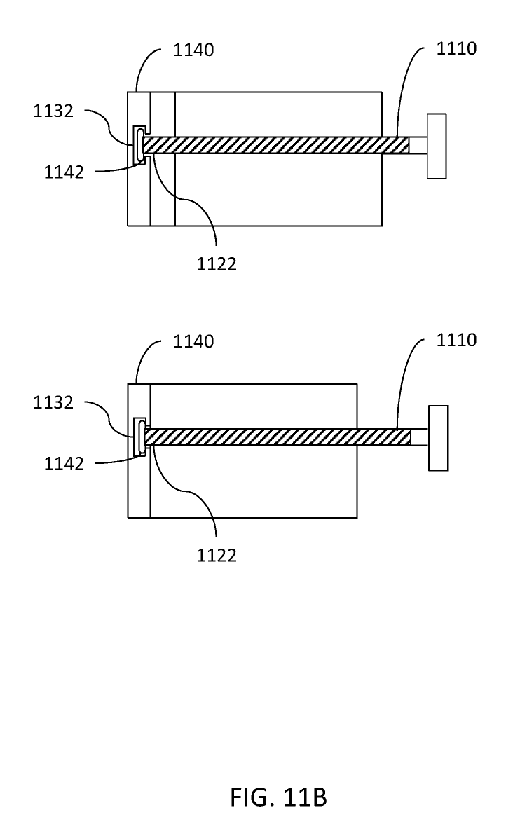

Another embodiment, illustrated in FIG. 11A (both perspective and front views) is similar to the implementation of FIG. 10 but differs, for example, in the design of the locking mechanism. As illustrated in FIGS. 11A and 11B, this exemplary implant holder 1100 can include a locking screw 1110 that is configured to engage side walls 1120 in a manner to prevent movement of the side walls.

An exemplary operation of such a locking screw is explained with reference to FIG. 11B. As shown, horizontal slot 1132 can be formed in an “end portion” of the side wall structure (labeled 1140 in the top portion of FIG. 11A). The locking screw 1110 can then be threaded through one of the side wall structures such that its tip 1122 (including a flange 1142) goes into horizontal slot 1132. When locking screw 1110 is not tightened, side walls 1120 can slide relative to each other (see top portion of FIG. 11B). When locking screw 1110 is tightened, flange 1140 pulls against horizontal slot 1130 causing side walls 1120 to frictionally engage and lock in place (see bottom portion of FIG. 11B).

In use, the side walls can be opened to sufficiently accept the implant, then closed to cause compression around the implant and stabilize it, then the locking screw can be screwed in to prevent the sidewalls from moving during the medical procedure.

The exemplary design of FIG. 11A also shows an optional feature where at least one of the side walls includes an end portion having a recess 1130 with a shape complementary to the implant. In the particular implementation depicted, the implant would be rounded in a similar manner to the rounded recess 1130.

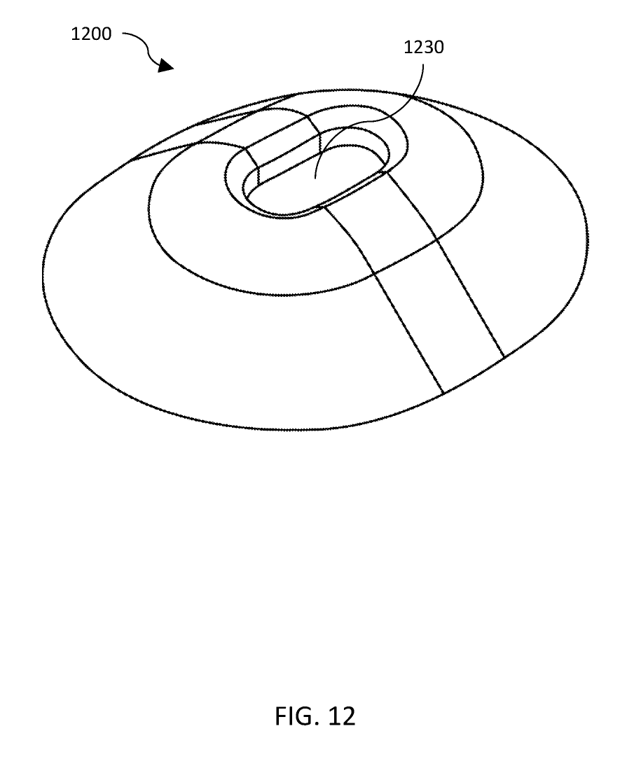

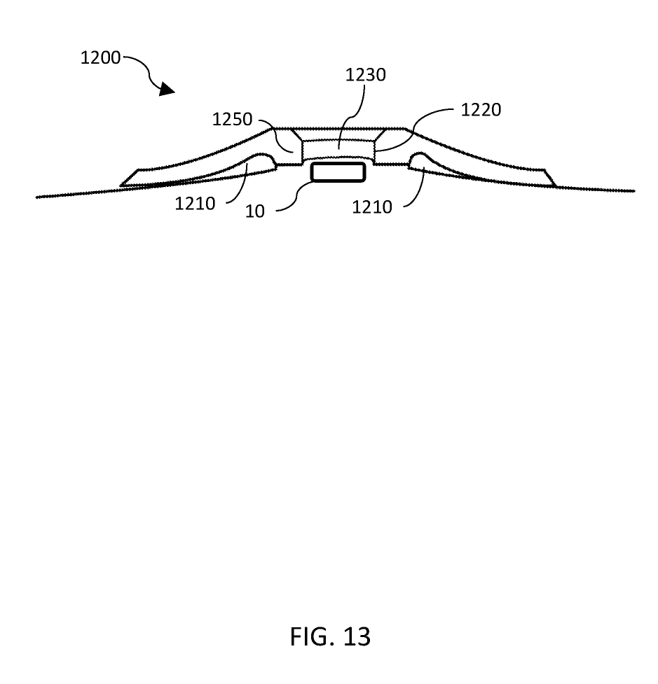

In an alternative embodiment, illustrated in FIGS. 12-14, implant holder 1200 can be designed similar to a suction cup where suction between the implant holder and the patient keeps the implant holder in place. With reference to FIG. 13, to allow the implant holder to function as a suction cup, the implant holder can be flexible and shaped to form space 1210 between the implant holder and the patient such that when the implant holder is at least partially flattened against a patient anatomy (e.g., by a patient pushing down on implant holder), vacuum forms in the space to hold the implant holder in place.

As with other implementations as described herein, this implementation of an implant holder for stabilizing an implant of a patient can include at least one implant-engaging surface 1220 configured to cause compression of skin adjacent the implant. Similar to other implementations described herein, there can also be an aperture 1230 formed at least partially by the implant-engaging surface. In addition, the aperture can have a complementary shape to the implant.

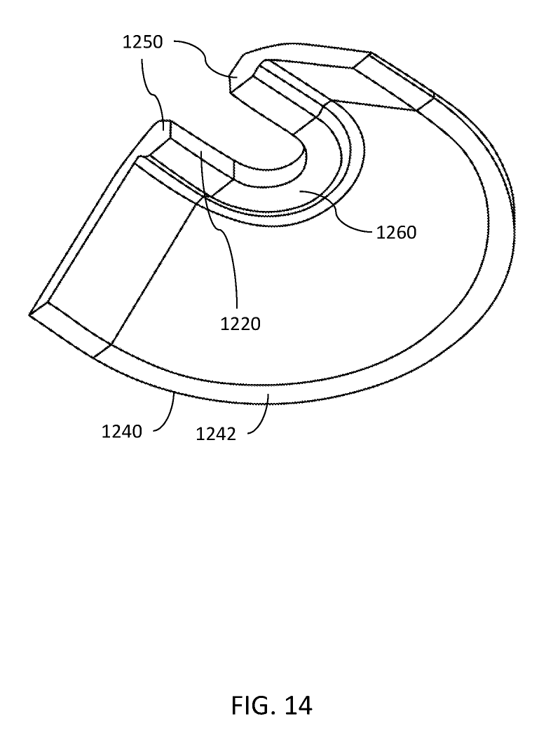

With reference to FIG. 14, the implant holder can also include an adhesive 1242 on at least a portion of a patient-facing surface near the perimeter 1240 of the implant holder. As seen in FIGS. 13 and 14, the implant holder can include side walls 1250 having implant-engaging surfaces 1220 and there can also be adhesive placed on at least a portion of the patient-facing surfaces 1260 of the side walls. The adhesives present on these respective surfaces can assist in maintaining the placement and or vacuum seal of the implant holder.

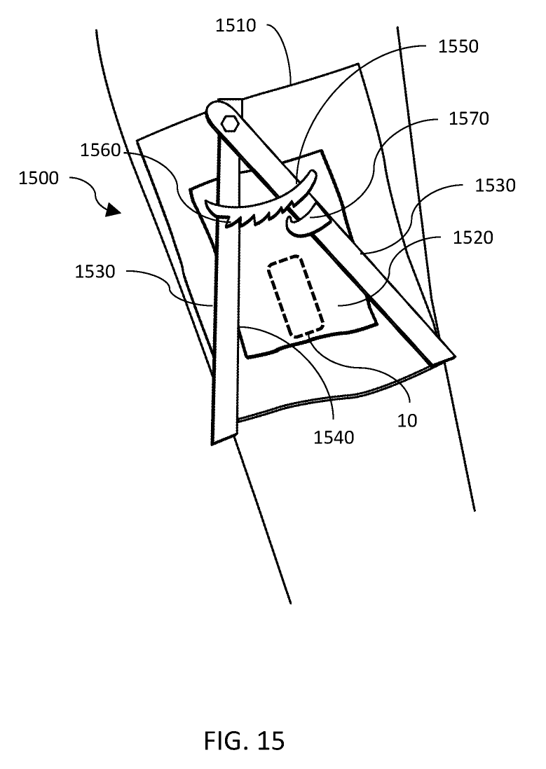

Other implementations of the technologies disclosed herein may include an adhesive patch for adhering the implant holder to the skin of the patient by the implant holder being coupled to the adhesive patch. Examples of such implementations are depicted in FIGS. 15-18. As shown, adhesive patches 1510 and 1710 can optionally have an opening (1520/1720) above the implant 10 to allow access to the implant without having to needle through the adhesive patch.

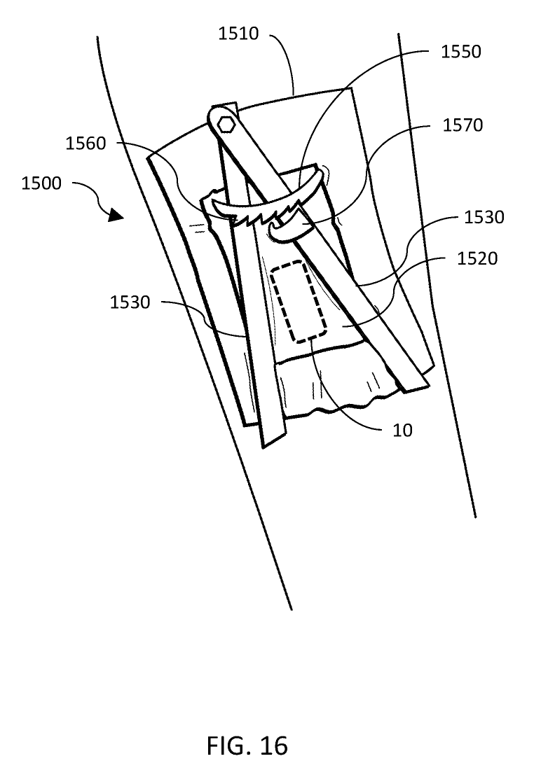

One exemplary implementation utilizing an adhesive patch is illustrated in FIGS. 15 and 16, showing an implant holder for stabilizing an implant of a patient, the implant holder including implant-engaging elements 1530 with at least one implant-engaging surface 1540. The implant holder can have an open configuration (as shown in FIG. 15) where the implant-engaging elements are disposed around opposing sides of the implant. The implant holder can also have a closed configuration (as shown in FIG. 16) where the implant-engaging elements can cause a compression of skin adjacent the implant that stabilizes the implant. The implant holder can also include a retaining mechanism that can maintain the compression.

In the exemplary implementation shown in FIGS. 15 and 16, retaining mechanism 1550 can include teeth 1560 and a pawl 1570. The pawl can be configured to engage the teeth to retain the closed configuration. Because there can be numerous teeth with any desired tooth density, the specific closed configuration can be set by a user to achieve a desired compression. Also, the implant holder can be further configured to transition from the closed configuration to the open configuration when the pawl is pulled back so as to not be engaged with the teeth. This can act as a quick-release mechanism in order to allow a user to remove the implant holder when desired.

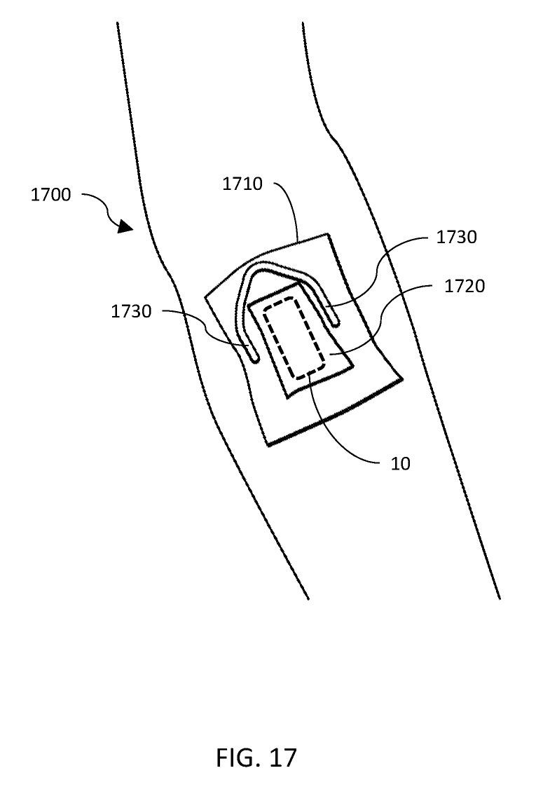

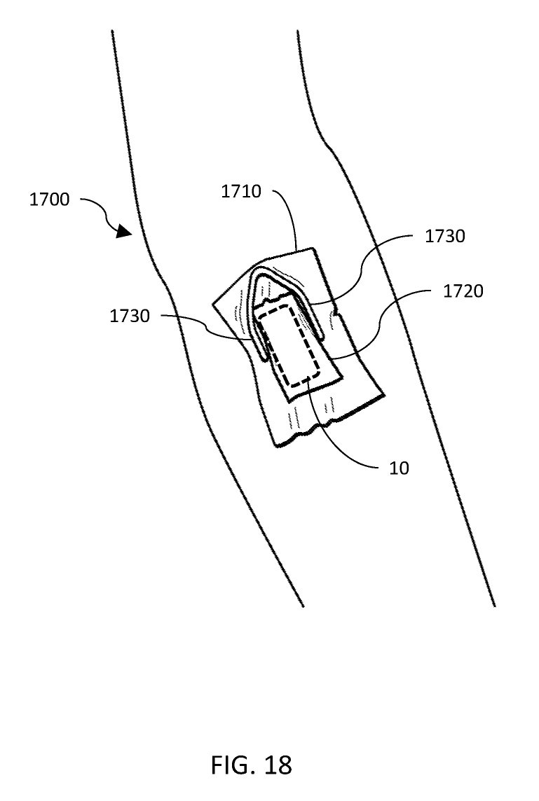

The implementation shown in FIGS. 17 and 18 is similar to the implementation of FIGS. 15 and 16 where implant holder 1700 includes implant-engaging elements 1730 that can be coupled to a patch 1710 having an opening 1720. However, the implant-engaging elements 1730 in this example can be formed of a material, such as a wire, shaped to allow compressive engagement of an implant when the implant holder is pinched around the implant. Specifically, such implementations of an implant holder 1700 can be configured to change from their open configuration (FIG. 17) to their closed configuration (FIG. 18) by the application of force and the plastic deformation of the implant holder. In these implementations, the retaining mechanism can be a property of the material (e.g., a metal) of the implant holder that will resist plastic deformation below, for example, 2.2, 4.4, 8.9, or 17.8 N (or 0.5, 1, 2, or 4 pounds) of force. In this way, the retaining mechanism can maintain the proper compression around the implant.

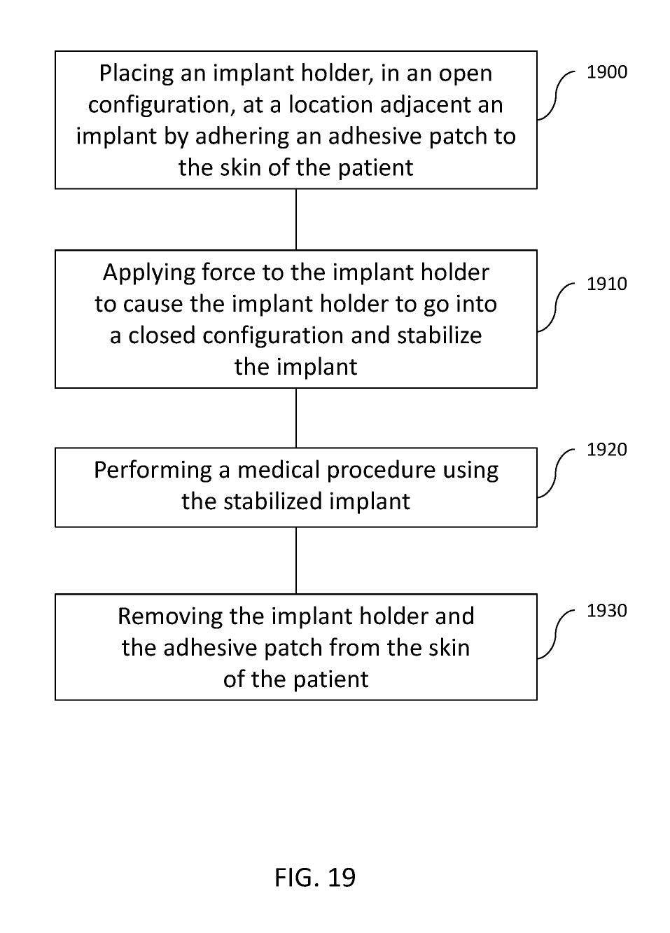

Implementations of these designs can be utilized in procedures performed by a patient themselves or with the assistance of a clinician. For example, exemplary method of use can utilize an implant stabilization apparatus having an implant holder including implant-engaging elements with at least one implant-engaging surface, the implant holder having an open configuration where the implant-engaging elements can be disposed around opposing sides of the implant and a closed configuration where the implant-engaging elements can cause a compression of skin adjacent the implant that stabilizes the implant, the implant holder further including a retaining mechanism that can maintain the compression and the implant holder coupled to an adhesive patch for adhering the implant holder to the skin of the patient. As shown in FIG. 19, the method of use can include, at 1900, placing the implant holder, in an open configuration, at a location adjacent the implant by adhering an adhesive patch to the skin of the patient.

At 1910, a force can be applied to the implant holder to cause the implant holder to go into the closed configuration and stabilize the implant.

At 1920, a medical procedure can be performed using the stabilized implant, kidney dialysis, for example.

At 1930, the implant holder and the adhesive patch can be removed from the skin of the patient.

Certain exemplary methods can also include the patient performing one or more of the placing of the implant holder on the skin, the performing of the medical procedure, and the removing of the implant holder from the skin.

In certain circumstances and implant holder may be used that includes a retaining mechanism with teeth and a pawl, the pawl configured to engage the teeth to retain the closed configuration, the implant holder further configured to transition from the closed configuration to the open configuration when the pawl is not engaged with the teeth. In some cases, the method can also include disengaging the pawl from the teeth to allow the implant holder to transition from the closed configuration to the open configuration.

While a particular exemplary method has been disclosed for the use of an implant holder in conjunction with an adhesive patch, other methods of use for different implant holder implementations discussed above are evident. For example, securing in place an implant holder using any of the holding mechanisms described above, causing an implant holder to exert a compressive force on an implant utilizing any of the compressive methods disclosed above, performing a medical procedure, and removing the implant holder from the patient.

Although the present disclosure details a number of specific embodiments, it is contemplated that many of the different technologies enumerated herein may be mixed and matched in order to provide additional implementations of the concepts disclosed herein.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods and/or articles depending on the desired configuration. Any methods depicted in the accompanying figures and/or described herein do not necessarily require the particular order shown, or sequential order, to achieve desirable results. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. The implementations described above can be directed to various combinations and subcombinations of the disclosed features and/or combinations and subcombinations of further features noted above. Furthermore, above described advantages are not intended to limit the application of any issued claims to processes and structures accomplishing any or all of the advantages.

Additionally, section headings shall not limit or characterize the invention(s) set out in any claims that may issue from this disclosure. Further, the description of a technology in the “Background” is not to be construed as an admission that technology is prior art to any invention(s) in this disclosure. Neither is the “Summary” to be considered as a characterization of the invention(s) set forth in issued claims. Furthermore, any reference to this disclosure in general or use of the word “invention” in the singular is not intended to imply any limitation on the scope of the claims set forth below. Multiple inventions may be set forth according to the limitations of the multiple claims issuing from this disclosure, and such claims accordingly define the invention(s), and their equivalents, that are protected thereby.

1. An apparatus for stabilizing an implant of a patient, the apparatus comprising:
an implant holder including at least one implant-engaging surface configured to cause compression of skin adjacent the implant; and
a strap assembly coupled to the implant holder and configured to tighten around patient anatomy in order to secure the implant holder in place above the implant.

2. The apparatus of claim 1, wherein the implant holder further includes side walls, the side walls having the implant-engaging surfaces.

3. The apparatus of claim 2, wherein at least one of the side walls includes an end portion with a recess having a shape complementary to the implant.

4. The apparatus of claim 2, wherein the implant holder further includes a tightening mechanism configured to cause movement of the side walls thereby adjusting a compressive force around the implant.

5. The apparatus of claim 4, the tightening mechanism including gear tracks coupled to the side walls and a gear configured to engage the gear tracks and cause the movement of the side walls.

6. The apparatus of claim 5, the tightening mechanism including a knob coupled to the gear and configured such that actuation of the knob causes the movement of the side walls.

7. The apparatus of claim 5, the tightening mechanism including a lock knob in communication with the gear and configured such that, when in a locked position, the lock knob locks the gear in place and prevents the movement of the side walls.

8. The apparatus of claim 2, further comprising a locking screw configured to engage the side walls in a manner to prevent the movement of the side walls.

9. The apparatus of claim 2, wherein the side walls are fixed.

10. The apparatus of claim 9, the implant holder further comprising an end wall disposed between the side walls for resisting an axial motion of the implant.

11. The apparatus of claim 9, the side walls forming a slot such that the implant holder is substantially open above the implant.

12. The apparatus of claim 1, the implant holder including an aperture with an inner surface having a complementary shape to the implant, the inner surface comprising the implant-engaging surface.

13. The apparatus of claim 2, wherein the implant holder is flexible and configured to at least partially conform to the patient anatomy when the strap assembly is tightened around the patient anatomy and further configured so that flexing of the implant holder causes movement of the side walls to increase a compressive force around the implant.

14. The apparatus of claim 1, the implant holder further comprising legs extending from the implant holder, the legs having feet at the ends of the legs, wherein the feet are configured to frictionally engage with the skin of the patient and pull the skin taut.

15. An implant holder for stabilizing an implant of a patient, the implant holder comprising:
at least one implant-engaging surface configured to cause compression of skin adjacent the implant; and
an aperture formed at least partially by the at least one implant-engaging surface,
wherein the implant holder is flexible and shaped to form a space between the implant holder and the patient such that when the implant holder is at least partially flattened against a patient anatomy, a vacuum forms in the space to hold the implant holder in place.

16. The implant holder of claim 15, wherein the aperture has a complementary shape to the implant.

17. The implant holder of claim 15, further comprising an adhesive on at least a portion of a patient-facing surface near a perimeter of the implant holder.

18. The implant holder of claim 15, wherein the implant holder includes side walls, the side walls having the implant-engaging surfaces.

19. The implant holder of claim 18, further comprising an adhesive on at least a portion of patient-facing surfaces of the side walls.

20-33. (canceled)
